(19) United States
(12) Patent Application Publication
(10) Pub. No.: US 2016/0190844 A1
(43) Pub. Date: Jun. 30, 2016

(54) OPTIMIZATION OF A BATTERY DRIVEN ULTRASOUND DEVICE

(71) Applicant: GENERAL ELECTRIC COMPANY, Schenectady, NY (US)

(72) Inventors: Trond Sandvik, Oslo (NO); Steinar Bjaerum, Horten (NO)

(73) Assignee: GENERAL ELECTRIC COMPANY, Schenectady, NY (US)

(21) Appl. No.: 14/584,205

(22) Filed: Dec. 29, 2014

(51) Int. Cl. H02J 7/00; A61B 8/00; G01R 31/36

(52) U.S. Cl. CPC H02J 7/0054; G01R 31/3606; A61B 8/4427; A61B 8/56

(57) ABSTRACT

An apparatus is described herein. The apparatus includes a first battery and a main battery. The first battery is a phone battery. The main battery is a battery of a handheld ultrasound system and the main battery is to maintain a charge of the first battery at an optimal charge level.

### BACKGROUND OF THE INVENTION

Portable ultrasound systems often include rechargeable batteries for ease of portability. Portable ultrasound systems enable ultrasound data to be quickly accessible in a variety of situations. For example, portable ultrasound systems can add more information to routine examinations where a traditional ultrasound device is not present. Moreover, portable ultrasound systems are useful in emergency situations, and can provide valuable information to medical service providers, such as emergency medical personnel.

Handheld ultrasound systems offer increased portability and maneuverability when compared to portable ultrasound systems. Often, handheld systems include one or more rechargeable batteries. As a result, the battery of the portable ultrasound system can be charged as necessary. When the ultrasound system includes a Smartphone, frequent charging and discharging of the Smartphone battery can reduce the lifespan of the Smartphone battery.

### SUMMARY OF THE INVENTION

An embodiment relates to an apparatus. The apparatus includes a first battery and a main battery. The first battery is a phone battery. The main battery is a battery of a handheld ultrasound system and the main battery is to maintain a charge of the first battery at an optimal charge level.

Another embodiment relates to a handheld ultrasound system. The handheld ultrasound system includes a Smartphone unit, an acquisition unit, and a main battery. The Smartphone unit includes a Smartphone battery. The acquisition unit includes power management functionality and is communicatively coupled with the Smartphone unit. The main battery is to provide power such that a charge level of the Smartphone battery is within an optimal range.

Still another embodiment relates to a method. The method comprises detecting a charge level of a first battery and determining if the charge level is within an optimal charge range of the first battery. The method also comprises charging the first battery from a second battery, wherein the second battery is to maintain the charge level of the first battery within the optimal range.

### BRIEF DESCRIPTION OF THE DRAWINGS

The present techniques will become more fully understood from the following detailed description, taken in conjunction with the accompanying drawings, wherein like reference numerals refer to like parts, in which:

FIG. 1 is a block diagram of a handheld ultrasound system;

FIG. 2 is a ladder diagram illustrating communication between a power management block and a phone;

FIG. 3 is a process flow diagram of a method for optimizing battery life; and

FIG. 4 is a process flow diagram of a method for optimizing battery life across a USB connection.

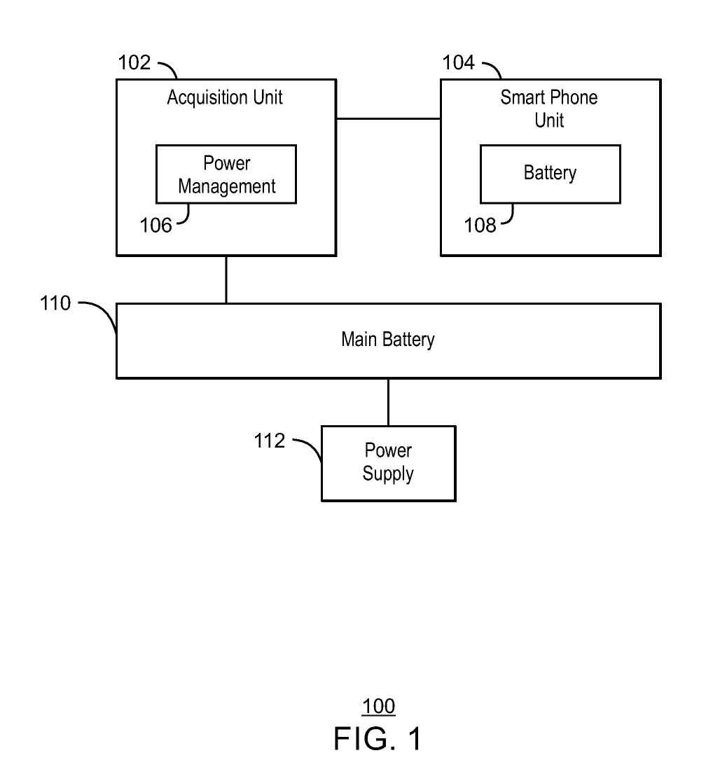

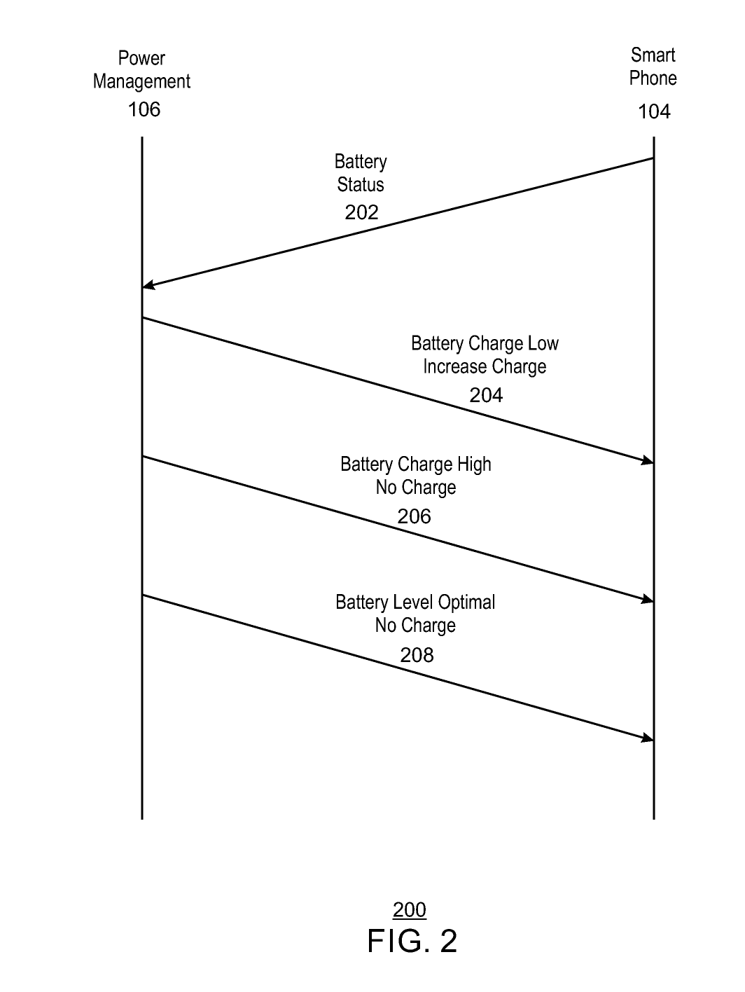

In some cases, the same numbers are used throughout the disclosure and the figures to reference like components and features. Numbers in the 100 series refer to features originally found in FIG. 1; numbers in the 200 series refer to features originally found in FIG. 2; and so on.

### DETAILED DESCRIPTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

Handheld ultrasound devices are designed to be charged and discharged may times over a life cycle. Many factors can affect the life cycle of a battery, including but not limited to ambient temperature, amount of charge (i.e., charging to 40% of capacity vs. 80% of capacity), and operating temperature. Thus, power management includes managing battery features in order to maximize the life cycle of the battery. In handheld ultrasound devices, poor power management often results in decreased effectiveness of the device. Specifically, the battery of the ultrasound device may reach then end of its lifespan at a much earlier time compared to other components of the device. Additionally, the battery of the ultrasound device may reach then end of its lifespan at a much earlier time compared to a battery with managed charge levels. A technical effect of the present techniques is a battery with consistent charging ability throughout its lifespan. Moreover, a technical effect of the present techniques is an increase in the lifespan of the battery.

FIG. 1 is a block diagram of a handheld ultrasound system 100. The handheld ultrasound system 100 includes an acquisition unit 102 and a Smartphone unit 104. In embodiments, the acquisition unit is a probe. Additionally, in embodiments, the Smartphone is an integrated component of the handheld ultrasound system 100. The probe may be connected to an enclosure including the Smartphone unit and other hardware to obtain and process ultrasound images via the probe. The Smartphone unit 104 may include a Smartphone battery 108. The Smartphone battery 108 is to power components of the Smartphone unit. Additionally, a main battery 110 may be included in the handheld ultrasound system 100. In embodiments, the main battery 110 is to power the acquisition unit 102. The main battery may also charge the Smartphone battery 108. The power management 106 is to perform power management functionality. For example, the power management 106 may include controlling the charging of the Smartphone battery 108, such that the charge level of the Smartphone battery is within a pre-determined range in order to ensure a maximum life cycle for the Smartphone battery 108.

A power supply 112 is to charge the main battery 110. In embodiments, power supply 112 is an external DC power supply. As used herein, an external DC power supply is located outside an enclosure that includes the acquisition unit 102, the Smartphone unit 104, and the main battery 110. The main battery may be charged by an internal charger coupled to an external DC power supply 112. In embodiments, the main battery 110 can charge the battery 108 at all times. Thus, the main battery 110 can charge the battery 108 when the handheld ultrasound system 100 is in operation. As used herein, in operation refers to a state of the handheld ultrasound system 100 where is powered on. During this powered on state, the handheld ultrasound system 100 may be used to obtain ultrasound images via the acquisition unit, or the handheld ultrasound system 100 may be used to display ultrasound images via the acquisition unit.

The main battery 110 can also charge the battery 108 when the handheld ultrasound system 100 is not in operation. Power management at the acquisition unit can then use power from the main battery 110 to ensure a charge level of the battery 108 is within an optimal level. Rechargeable batteries, such as the battery 108, are charged many times over the battery's lifespan. Typically, the battery is charged to a same fixed charge voltage each time that the battery is recharged. This fixed charge voltage is usually a maximum charge so that the length of time the battery supports operation is maximized. When a battery supports operation, components powered by the battery are powered on or drawing power from the battery. Charging the battery to a maximum charge, then discharging the battery without any power management may result on a shorter lifespan for the battery. Accordingly, the present techniques enable a power management such that charge levels of the battery are optimized in order to maximize a lifespan of the battery. Power management block 106 is to perform power management functions. In particular, the power management block 106 is to manage the charging capacity of the battery 108, such that the charging of the battery is optimized. In embodiments, the power management block 106 is an intelligent charger.

In embodiments, power management includes an algorithm to ensure a charge level of the Smartphone battery is within an optimal range. The optimal range may depend on the type of battery, the ambient temperature surrounding the battery, and so on. In embodiments, the main battery or the phone battery may be a Lithium-Ion battery (Li Ion), Nickel Cadmium (NiCd), Nickel Metal Hydride (NiMH), or Sealed Lead Acid (SLA) batteries. An optimal charge range of the battery is determined. In embodiments, the optimal charge range is based on the type of battery. The optimal charge range may also be based on manufacturer's recommended charge levels. Moreover, the optimal charge range may also be based on charge levels determined via battery tests.

FIG. 2 is a ladder diagram illustrating communication between a power management block 106 and a Smartphone unit 104. The Smartphone unit 104 may transmit a battery status to the power management block 106 at reference number 202. The battery status may include, but is not limited to, a level of charge of the battery, a temperature of the battery, and the like. In embodiments, the power management block 106 may poll the Smartphone unit 104 for battery status information. The battery status may also be transmitted to the acquisition unit over a proprietary protocol on top of the Android Open Accessory (AOA) protocol. The AOA protocol enables USB hardware to interact with an Android-powered device in a special accessory mode. In this manner the acquisition unit can function as a USB host, while the Smartphone unit 104 is an Android-powered device. According to the AOA protocol, the acquisition unit is to provide 500 mA at 5 V for charging the Smartphone unit 104. In embodiments, the Smartphone 104 may initiate contact with the acquisition unit via the AOA protocol. Additionally, in embodiments, the proprietary protocol is to package the battery status information and send the battery status information to the acquisition unit according to the proprietary protocol.

Upon the receipt of the battery status information at reference number 202, the power management block can manage a charge level of the phone battery based on the received battery status information. In particular, if the battery charge is low, the power management block can increase the charge of the battery at reference number 204. If the battery charge is high, the power management block does not apply any charge to the smart phone 104. If the battery charge level is optimal, the power management block does not apply any charge to the smart phone 104.

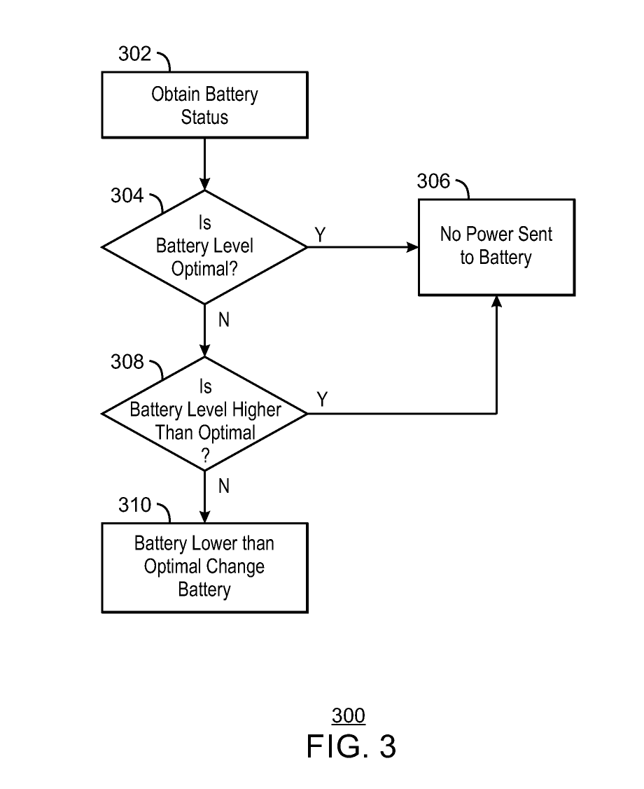

FIG. 3 is a process flow diagram of a method 300 for optimizing a battery lifespan. At block 302, the battery status is obtained. The battery status can be obtained using a number of protocols, including a Peripheral Component Interconnect (PCI) Express (PCIe) Specification, such as the PCIe 3.0 released on Nov. 10, 2010; a Universal Serial Bus (USB) Specification, such as the USB 3.1 Specification released on Jul. 26, 2013, or a Serial ATA (SATA) Specification, such as the SATA 3.2 Specification released in August 2013. At block 304, it is determined if the battery charge level is optimal. As used herein, an optimal battery charge level is a level of battery charging capacity that is to enable a maximum life span of the battery. In some embodiments, this level is expressed as a percentage of the total charging capability of the battery. Additionally, the optimal charge level can be a range of battery charging levels that result in a maximum lifespan of the battery. For example, the maximum charge level may be 70%-80% of the total battery charging level. In embodiments, additional information may be used to determine the optimal charge level of the battery.

If the battery level is optimal, process flow continues to block 306. If the battery level is not optimal, process flow continues to block 308. At block 306, no power is sent to the phone battery. At block 308, it is determined if the battery level is higher than optimal. If the battery level is higher than optimal, process flow continues to block 306 where no power is sent to the phone battery. If the battery level is lower than optimal, the battery is charged. The battery may be charged by controlling the current levels provided to the Smartphone by the acquisition unit. The battery status may be obtained periodically while the Smartphone battery is charging. The battery may also charge while the handheld ultrasound device is in operation. While charging is described as being performed via the main battery, in some embodiments, the Smartphone battery may be charged via an external intelligent charger connected directly to the Smartphone unit via a dedicated external receptacle, wherein the receptacle is external to a handheld ultrasound unit including the acquisition unit, main battery, and Smartphone unit.

Battery management information may be transmitted via the Universal Serial Bus protocol. In examples, the acquisition unit may function as a host, while the phone functions as a device. The host can be used to charge the device according to the USB Specification. Moreover, in embodiments, the power management of the handheld ultrasound device may be performed according to the USB Power Delivery Specification, Revision 2.0, released Aug. 11, 2014.

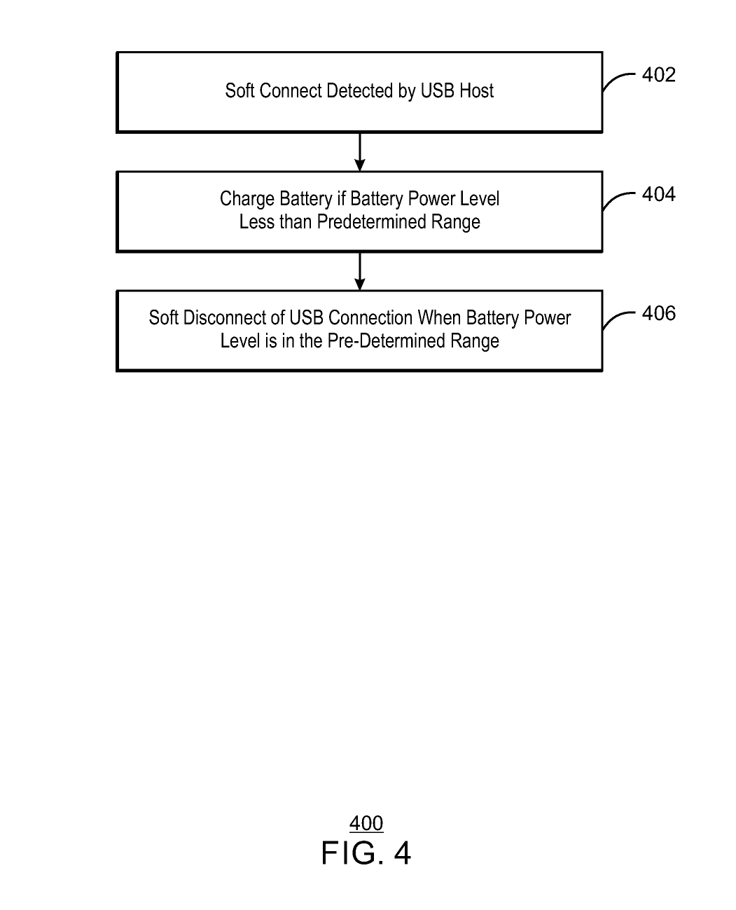

FIG. 4 is a process flow diagram of a method 400 for optimizing the battery life across a USB connection. At block 402, a soft connect is detected by the USB host. A soft connect is a connection between two or more components according to a USB protocol via the execution of a command. In embodiments, the soft connect command may be generated by a microcontroller of the host or a device. In embodiments, the USB host is an acquisition unit of the handheld ultrasound device, while the Smartphone is a USB device. At block 404, the battery of the device is charged if a power level of the battery is not in a predetermined range. The pre-determined power range is range of power levels of the battery, where such power levels can extend a lifespan of the battery. At block 406, a soft disconnect of the USB connection is performed when the battery power levels are within the pre-determined range. A soft disconnect is a disconnect of the connection between two or more components according to a USB protocol via the execution of a command.

By the Smartphone battery in a managed, adaptive manner, a lifespan of the battery may be extended while still providing a portable, powered device that with a charge that lasts lone enough to meet user's expectations. The present techniques extend the battery lifetime compared to a non optimized battery charging regime. Further, customers can use the handheld ultrasound device for a longer period of time before noticing reduced battery capacity. This results in lower service costs of the device.

The various embodiments are not limited to medical imaging systems for imaging human subjects, but may include, for example, veterinary systems. As used herein, the term “patient” may refer to a human patient or any other animal.

While embodiments are described herein with respect to modality units used in the medical field, embodiments described herein can encompass those situations in which any modality unit is used in an imaging procedure. Further, those of skill in the art will recognize that the present techniques are applicable to many different hardware configurations, software architectures, organizations, or processes.

While the detailed drawings and specific examples given describe particular embodiments, they serve the purpose of illustration only. The systems and methods shown and described are not limited to the precise details and conditions provided herein. Rather, any number of substitutions, modifications, changes, and/or omissions may be made in the design, operating conditions, and arrangements of the embodiments described herein without departing from the spirit of the present techniques as expressed in the appended claims.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An apparatus, comprising:
a first battery, wherein the first battery is a phone battery; and
a main battery, wherein the main battery is a battery of a handheld ultrasound system and the main battery is to maintain a charge of the first battery at an optimal charge level.

2. The apparatus of claim 1, wherein the optimal charge level is a level of battery charging capacity that is to enable a maximum life span of the battery.

3. The apparatus of claim 1, wherein the optimal charge level is 70%-80% of the total battery charging level.

4. The apparatus of claim 1, wherein the main battery is to maintain a charge of the first battery at an optimal charge level during operation of the handheld ultrasound system.

5. The apparatus of claim 1, wherein the optimal charge level ensures a maximum life cycle of the first battery.

6. A handheld ultrasound system, comprising:
a Smartphone unit, wherein the Smartphone unit includes a Smartphone battery;
an acquisition unit, wherein the acquisition unit includes power management functionality and is communicatively coupled with the Smartphone unit; and
a main battery; wherein the main battery is to provide power such that a charge level of the Smartphone battery is within an optimal range.

7. The handheld ultrasound system of claim 6, wherein the optimal range is a range of battery charging capacity that is to enable a maximum life span of the battery.

8. The handheld ultrasound system of claim 6, wherein the main battery is to be charged by an internal charger coupled to a DC power supply.

9. The handheld ultrasound system of claim 6, wherein the acquisition unit is communicatively coupled with the Smartphone unit according to a Universal Serial Bus protocol.

10. The handheld ultrasound system of claim 6, wherein the acquisition unit provides power to the Smartphone battery according to a Universal Serial Bus protocol.

11. The handheld ultrasound system of claim 6, wherein the power management functionality is to perform a soft connect to the Smartphone unit to obtain Smartphone battery status information from the Smartphone unit.

12. The handheld ultrasound system of claim 6, wherein a Smartphone battery status is to be communicated to the acquisition unit using a proprietary protocol on top of an Android Open Accessory (AOA) protocol.

13. The handheld ultrasound system of claim 6, wherein the main battery is to maintain a charge of the Smartphone battery at an optimal charge level while the handheld ultrasound system is powered on.

14. The handheld ultrasound system of claim 6, wherein the Smartphone battery is charged via an external intelligent charger connected directly to the Smartphone unit via a dedicated external receptacle.

15. The apparatus of claim 1, comprising dual acquisition units.

16. A method, comprising:
detecting a charge level of a first battery;
determining if the charge level is within an optimal charge range of the first battery; and
charging the first battery from a second battery, wherein the second battery is to maintain the charge level of the first battery within the optimal range.

17. The method of claim 16, wherein the optimal charge range is to ensure a maximum lifespan of the battery.

18. The method of claim 16, wherein the first battery is a battery of a Smartphone.

19. The method of claim 16, wherein the second battery is a main battery of a handheld ultrasound system.

20. The method of claim 16, wherein an external DC power supply is to power the main battery.
